(19) United States
(12) Patent Application Publication
(10) Pub. No.: US 2008/0091083 A1
(43) Pub. Date: Apr. 17, 2008

(54) ANESTHETIC DRUG MODEL USER INTERFACE

(76) Inventors: Laurence M. Yudkovitch, Milwaukee, WI (US); Ronald P. Makin, Fitchburg, WI (US); Rene Coffeng, Helinski (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/563,993

(22) Filed: Nov. 28, 2006

Related U.S. Application Data
(60) Provisional application No. 60/851,108, filed on Oct. 12, 2006.

(51) Int. Cl. G06F 19/00; A61B 5/00

(52) U.S. Cl. 600/300; 702/19

(57) ABSTRACT

A graphical user interface for the documentation of an administered anesthetic drug and the display of an associated pharmacokinetic model and an associated pharmacodynamic model. The graphical user interface comprises a first window that displays the drug administration data, and a second window displaying a pharmacokinetic model and a pharmacodynamic model, the second window being separate and distinct from the first window. The pharmacokinetic model and the pharmacodynamic models are overlaid and displayed and the pharmacokinetic model and the pharmacodynamic model are based on the drug administration data displayed in the first window.

### CROSS REFERENCE TO RELATED APPLICATION

This application claims priority under 35 USC §119(e) of the co-pending U.S. Provisional Application 60/851,108, filed on Oct. 12, 2006 and entitled “ANESTHETIC DRUG MODEL USER INTERFACE.”

### FIELD OF THE INVENTION

The present invention relates to a user interface for a life support system, more specifically, the present invention relates to a user interface for an anesthetic drug model display.

### BACKGROUND OF THE INVENTION

In the operating room, the anesthesiologist needs to assess the patient's condition and adjust the therapy using a wide variety of distinct medical devices. These devices often have limited communication abilities between each other, resulting in an incomplete depiction of the patient's condition to the anesthesiologist. A clinician must therefore mentally keep track of the patient's level of sedation, analgesia, and relaxation, the three physiological components of anesthesia, based on the clinician's recall of the drugs that have been administered, and the clinician's own familiarity with each drug's pharmacokinetic (PK) and pharmacodynamic (PD) models.

The practice of intra-operative anesthesia typically involves administering sedative, analgesic, and neuromuscular relaxant drugs or agents to a patient. These drugs manage the patient's level of consciousness, pain management, and neuromuscular blockade. Typically, each drug has a pharmacokinetic model that specifies what the body does to the drug and a pharmacodynamic model that specifies how the drug interacts with the body. More specifically, the pharmacokinetic model represents how the drug is absorbed, distributed, and broken down by the patient's body. The pharmacodynamic model approximates the effect that the patient feels from the administration of the drug. These models are usually derived in isolation from one another based upon standard demographic information of the patient such as sex, age, height, and weight. However, in a clinical setting, multiple drugs are typically used together. The interactions between these drugs may be additive, and produce no additional effects, may be synergistic, and produce a greater total effect than the sum of the individual drug effects, or may be antagonistic, and produce less total effect than the sum of individual effects.

The interaction between two anesthetic drugs has been represented by three-dimensional response surfaces. These surfaces represent the probability of a non-response to a specific effect at different concentrations of the two drugs. However, these three-dimensional graphs are complex and difficult to display on a display that is typically available for the display of a graph based on an anesthetic drug model. Therefore, the challenge is to display these varying probabilities of drug interactions on a two-dimensional graph that can easily be interpreted by a clinician during anesthesia.

The display of drug pharmacokinetics and the resulting pharmacodynamics becomes still more complex when more than one pharmacodynamic effect must be displayed on the same graph. For example, when considering analgesia, one can consider varying levels of pain such as high pain (intubation) and low pain (post-op). The challenge is to display these related, but distinct effects on the same two-dimensional graph. Therefore, it is desirable that the display be able to display the pharmacokinetic information of the effect site concentration of the administered drugs, at least one pharmacodynamic effect, the probability of each displayed effect, and reference points to the probability range of those effects.

Displays have been developed in the prior art that show both the PK and PD graphs to the clinician in a real time display. This display, however, is limited in its ability as a clinical user interface. The prior art display presented the drug administration data and the PK graph on a single graph, while displaying the PD graphs on a separate graph. This display of the relevant information is not an intuitive display as the PD effects experienced by the patient are dependent upon the effect site concentrations of the drugs as displayed in the PK graphs. Furthermore, if a clinician wants to identify each of the drug administrations that have taken place, the clinician must search each of the PK graphs to find the drug administration data related to that graph and compile this data.

The prior art display is further limited as the prior art display only supports the generic name of drugs, which makes it difficult for clinicians who aren't always familiar with generic names, or often rely upon the common names of the drugs. Additionally, the prior art display required the user to manually enter the concentration of the drug administered each time. This makes the system more difficult to use by clinicians who administer different concentrations of drugs throughout the same case. Further, the prior art display required that data be input in real time along with the display. This often resulted in the need for an extra clinician to be required in the operating room to manually document and enter the administration of anesthetic agents into the display in real time.

Therefore, it is desirable in the present field of anesthetic drug model displays to provide a user interface that improves display of the drug administration data and the PK and PD graphs. It is further desirable to provide a user interface that improves the ease with which the clinician can enter drug administration information, allows for the editing of the drug administration information retroactively, and addresses the scaling problems experienced in the prior art for the drug effectiveness ranges and the display of both PK and PD models on the same graph.

### SUMMARY OF THE INVENTION

A user interface for a pharmacokinetic and pharmacodynamic anesthetic drug model display is herein disclosed. In an embodiment, the user interface displays a window with drug administration data separate and distinct from windows displaying graphs of pharmacokinetic models and pharmacodynamic models of the drugs administered.

In an embodiment the user interface allows the editing of the drug name, drug concentration, the infusion rate or bolus amount, and administration time, or the deleting of an erroneous entry.

In a further embodiment of the user interface the clinician is able to enter drug administration data retroactively, defining the time at which the drug was administered.

In another embodiment of the user interface the clinician can activate a detailed information pop-up, the pop-up displaying additional information regarding drug administration, a PK graph, or a PD graph.

In another embodiment of the user interface upon initialization of the user interface, the user interface displays a pre-op warning to the clinician.

### BRIEF DESCRIPTION OF THE DRAWINGS

In the drawings:

FIG. 1 is a screenshot of an embodiment of the user interface.

FIG. 2 is a screenshot of an embodiment of the user interface.

FIG. 3 is a schematic diagram of an embodiment of a system comprising the user interface.

### DETAILED DESCRIPTION OF THE INVENTION

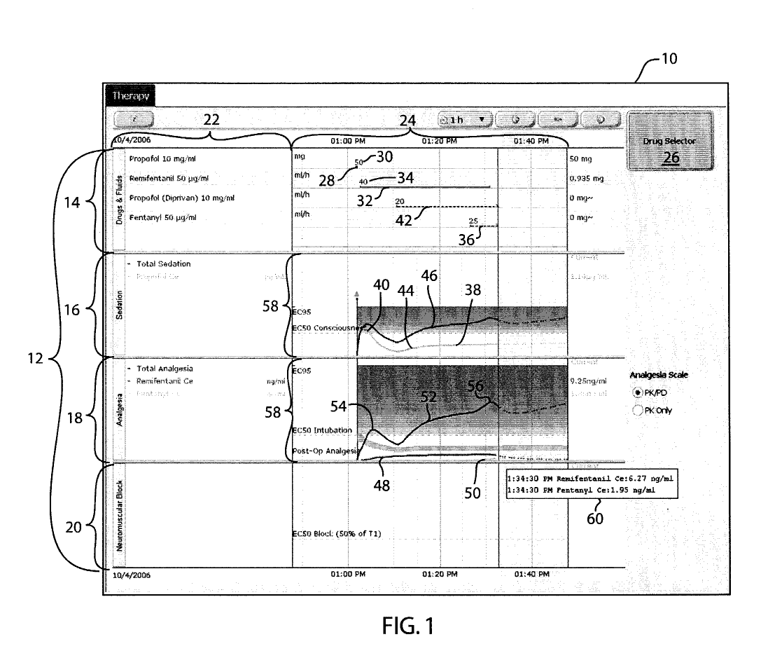

FIG. 1 depicts an embodiment of a user interface 10 for displaying drug administration documentation data and graphs based on the pharmacokinetic (PK) models and the pharmacodynamic (PD) models of the administered drugs. The user interface 10 may be displayed by any display or display associated with a device that may be present in a close proximity to a clinician providing anesthetic agents to a patient. Such a display may be a terminal for a computer workstation and may comprise CRT or flat-screen technology. Furthermore, the display may be such that the clinician interacts with the user interface 10 using touch-screen technology that is activated by the clinician's finger or a stylus, or alternatively the clinician controls a cursor with a mouse, data knob, or a directional pad. The user interface 10 comprises a plurality of windows 12. Embodiments of the user interface 10 may include a drug administration window 14, a sedation window 16, an analgesia window 18, and a neuromuscular block window 20. Each of the windows may comprise two regions, a drug listing region 22 and a time-based graph region 24.

A clinician may enter drug administration data by selecting a drug selector button 26. Alternatively, the clinician may enter drug administration data by selecting the drug administration window 14. The drug selector button 26 may open a drug library (not depicted) that can be edited by the hospital to include the potential anesthetic drugs that could be administered to a patient. Also, the drug library may include the drug concentrations that are available. Many anesthetic drugs have been studied to develop pharmacokinetic and pharmacodynamic models for the drugs based on basic patient demographic information such as age, sex, height, and weight. The drug library may include an indication of which drugs in the library have associated PK and PD models. Anesthesia can be divided into three basic effects: sedation (patient consciousness), analgesia (patient pain blocking), and neuromuscular blocking (patient relaxation). Each of the drugs in the drug library has a defined primary anesthetic effect. However, it is understood that any of the drugs may have effects in the other areas of anesthetic effect besides the drug's primary effect.

Once a clinician has selected a drug administration to document, the clinician may be prompted to enter the amount of the drug that was administered and the time at which the drug was administered. Further, the clinician may indicate that the administration was in the form of an injected bolus or as an IV infusion. Alternatively, if the device that is displaying the user interface 10 is connected to an IV infusion pump (not depicted) and the devices are able to properly communicate with each other the user interface may obtain drug infusion data such as the infusion rate and the infusion start and stop times from the infusion pump.

The user interface 10 displays the drug administration data in the drug administration window 14. The name of the drug appears in a listing in the drug listing region 22 of the drug administration window 14. As the administration of additional drugs are documented, these drugs are added to the bottom of the list. In an embodiment of the user interface 10, if more drugs have been administered than there is room for the drug administration window 14 to display, a scroll bar (not depicted) may appear, allowing a clinician to scroll through all of the documented drug administrations.

Additional drug administration data associated to the drugs listed in the drug listing region 22 is displayed in the graph region 24 of the drug administration window 14. For example, the drug Propofol is listed in the drug listing region 22. The number next to the name Propofol, “10 mg/ml” identifies the concentration of Propofol delivered. In the graph region 24 a dot 28 indicates a bolus of Propofol was delivered at approximately 1:02 PM. A drug amount indicator 30 identifies that 50 mg of Propofol was delivered in the bolus. Alternatively, the drug Remifentanil is listed in the drug listing region 22 at a concentration of 50 ug/ml. In the graph region 24, a line 32 indicates that an infusion of Remifentanil was delivered at approximately 1:03 PM. A drug rate indicator 34 identifies that the infusion was at a rate of 40 ml/hour. The line 32 is a solid line, thus indicating that the infusion has been completed, in this case the infusion represented by line 32 ended at approximately 1:31 PM. A dotted line, such as line 36 indicates that an infusion is presently ongoing, as the infusion of the drug Fentanyl indicated by line 36.

An embodiment of the user interface 10 allows for the documentation of a drug administration retroactively. This means that the clinician is allowed to enter the time of a drug administration when documenting a drug administration rather than the drug administration being only recorded in real time when the clinician documents it. Furthermore, once a drug administration has been documented by the clinician, the clinician can edit the drug administration data to correct any mistakes in the documentation, or to update the information, such as recording when an infusion of a drug ends.

The user interface 10 displays the proper pharmacokinetic (PK) models and pharmacodynamic (PD) models for each of the drugs documented by the clinician appearing in the drug administration window 14. The pharmacokinetic models and the pharmacodynamic models are displayed on a graph separately from the drug administration window 14. In an embodiment of the user interface 10, the user interface 10 further comprises the sedation window 16, the analgesia window 18, and the neuromuscular block window 20. As previously stated, each drug is classified as to the primary anesthetic effect of the drug. This primary anesthetic effect determines which window the PK and PD models for each drug are depicted.

The sedation window 16 displays the PK graph based on the PK model for any drugs that have a primary anesthetic effect as a sedative. Propofol has a primary effect as a sedative; therefore, Propofol is listed in the drug listing region 22 of the sedation window 16. The Propofol PK graph 38, displaying the effect site concentration of Propofol, is then displayed in the graph region 24 of the sedation window 16. The Propofol PK graph 38 is affected by the amount of Propofol administered, the time the Propofol was administered, and the characteristics of the Propofol PK model. Therefore, an initial spike 40 in the Propofol effect site concentration appears shortly after the administration of the 50 mg bolus 28, followed by decay until the initiation of the 20 ml/hr infusion of Propofol 42, which produces an increase 44 in the effect site concentration of Propofol.

The sedation window 16 also displays a sedation PD graph 46 based on a sedation PD model for any drugs that have an anesthetic effect as a sedative. The sedation PD graph 46 may include data from drugs that have also been delivered to the patient that are not a sedative in primary effect, but may still produce some sedative effect. As a result the sedation PD graph 46 is an indication of the total sedation of the patient.

The analgesia window 18 also displays the PK graphs based on the PK models for any drugs that have a primary anesthetic effect as an analgesic. In FIG. 1, both Remifentanil and Fentanyl have a primary effect as an analgesic, therefore the analgesia window 18 displays more than one PK graph simultaneously. Both a Remifentanil PK graph 48 and a Fentanyl PK graph 50 appear in the graph region 24 of the analgesia window 18.

The analgesia window 18 also displays an analgesia PD graph 52 based on an analgesia PD model for any drugs that have any anesthetic effect as an analgesic. Therefore, despite Propofol having a primary effect as a sedative, Propofol also produces or contributes to an analgesic effect, and as such, a spike 54 in the analgesia PD graph 52 appears coinciding with the initial introduction of the 50 mg bolus 28 of Propofol. Furthermore, the analgesia PD graph 52 represents the combined analgesic effect of all of the administered drugs, therefore another spike 56 appears when the infusion of Fentanyl 36 is administered to the patient.

The graph region 24 of both the sedation window 14 and the analgesia window 16 comprise a normalized scale 58. The normalized scale 58 represents the percentage of the population that experiences a sedation or analgesic effect at a particular sedation or analgesia level. The normalized scale 58 then marks the level at which 50% (EC50) and 95% (EC 95) of the population experience the sedation or analgesic effect. The normalized scales 58 for sedation and analgesia PD graphs are specific to the sedation and analgesia PD models respectively. Additionally, the PK graphs are normalized to the normalized scale 58 also. The PK graphs are normalized to the effect site concentration required for an administration of that drug only to achieve the same sedation or analgesia effect.

While FIG. 1 does not display any information in the neurological block window 20, a similar display of neuromuscular PK and PD graphs, as described for the sedation window 14 and the analgesia window 16 is contemplated and considered to be within the scope of the present invention.

An embodiment of the user interface 10 further comprises a detailed information pop-up 60. A clinician using the user interface 10 in association with a device or display comprising an input means such as touch-screen technology or a cursor that is controlled by a input means such as a mouse, data knob, directional pad, or a keyboard can activate the detailed information pop-up 60 by touching or placing the cursor over any portion of a PK graph. The detailed information pop-up 60 appears on the user interface 10 and comprises detailed information regarding one or more PK graphs. The detailed information pop-up may identify a drug and present timing data and effect site concentration data for that drug at that point in time. Alternatively, another detailed information pop-up (not depicted) may similarly operate to provide detailed information regarding the PD graphs or drug administration data in the drug administration window.

The detailed information pop-up 60 improves the user interface 10 by keeping the windows 12 simple and easy to interpret, but allowing the clinician to have access to more detailed information regarding a particular portion of a PK or PD graph or a drug administration. The detailed information pop-up 60 also provides a solution to the tendency of the normalized scale 58 to suppress the PK graph in relation to the scale. The clinician can use the detailed information pop-up 60 to receive a more precise reading of effect site concentration than received by visual inspection of the PK graph.

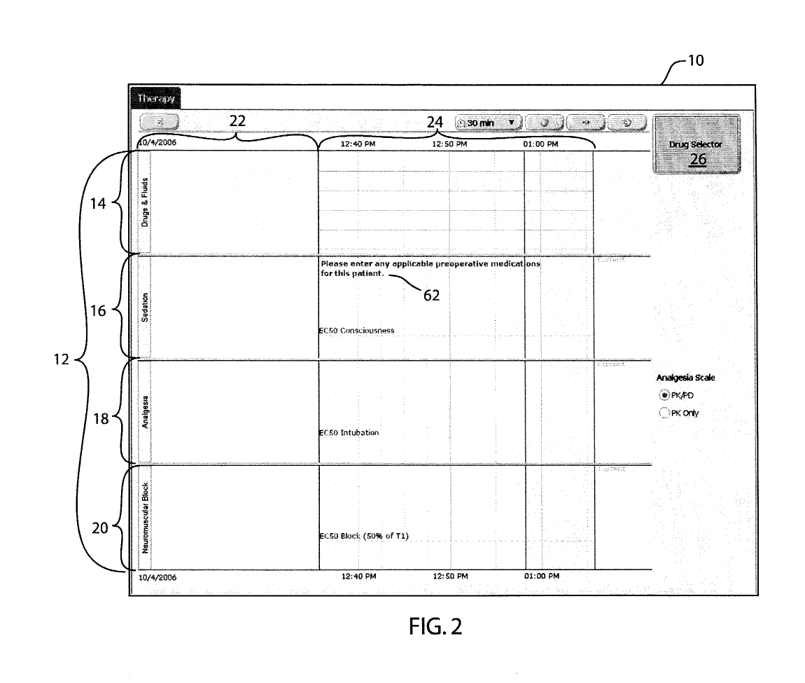

FIG. 2 depicts an embodiment of the user interface 10 as it may appear upon initialization of a program operating the user interface 10, before a clinician has documented the administration of any anesthetic drugs. The user interface 10 displays a pre-op warning message 62 located in the sedation window 16 that reminds the clinician to first enter into the user interface 10 any applicable preoperative medications that have already been administered to the patient. Alternatively, the pre-op warning message 62 may take the form of a text box or a textual message located in any of the other windows 12 of the user interface 10. This is an important safety feature because the clinician may forget to enter the preoperative medications that have been administered to the patient because the administration has already occurred. However, the presence of preoperative medications in the patient's body may have important effects on the PK or PD graphs for the drugs administered during the procedure.

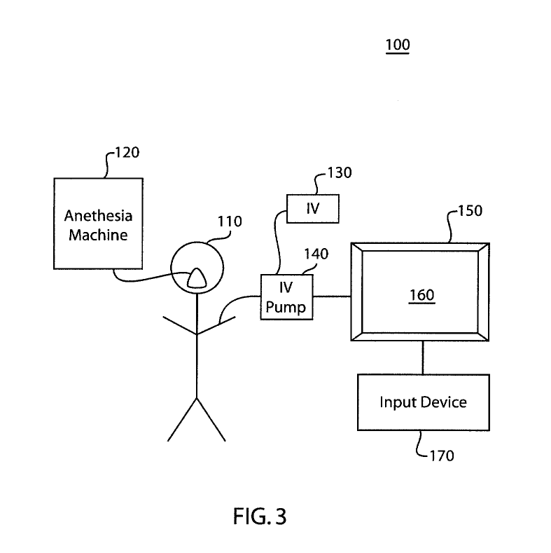

FIG. 3 depicts a schematic diagram of an embodiment of a system, such as a critical care system 100. The critical care system 100 comprises a patient 110. The patient 110 may be receiving an anesthetic agent from an anesthesia delivery machine 120, or via an intravenous (IV) drug delivery system 130. Additionally, the IV drug delivery system 130 may be connected to an IV pump 140. The IV pump 140 may be used to control the rate and amount of the drug delivered to the patient 110 by the IV drug delivery system 130. If the IV pump 140 is able to communicate with a display 150, the display 150 can receive IV drug administration data from the IV pump 140. The display 150 comprises a user interface 160 that displays drug administration data and PK and PD models to a clinician. Drug administration data not received by the display 150 from the IV pump 140 must be entered manually by the clinician. The clinician may enter the drug administration data into the display 150 using an input device 170. The input device 170 may be an external input device 170, such as a keyboard. Alternatively, the input device may be an input device that is integral with the display 150, such as a touch screen.

Embodiments of the user interface 10 enhance the ease of use and understandability of the drug administration data and PK and PD graphs displayed on the user interface 10. A separated drug administration window 14 from the other windows displaying PK and PD graphs allows for the clinician to more easily track the documentation of drug administrations and provides the clinician with a clear record of what drugs were administered, when drugs were administered, how much drug was administered, and how the drug was administered.

Furthermore, the documentation of drug administration and the quality of the PK and PD models are improved in embodiments of the user interface 10. In embodiments of the user interface 10, the clinician is able to document the administration of drugs retroactively. This eliminates the need for an additional clinician to be present to perform the task of documenting the administration of drugs in real time, as required by user interfaces in the prior art. This has the added effect of helping to reduce the crowding that may be experienced in an operating room by reducing the number of people in the room by one. Additionally, if the clinician notices an error in the documentation of the administration of a drug, the clinician can edit the drug administration data to correct the error. The error correction ability improves the quality of the PK and PD graphs that are displayed as the PK and PD graphs are dependent upon the drug administration data.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Various alternatives and embodiments are contemplated as being with in the scope of the following claims, particularly pointing out and distinctly claiming the subject matter regarded as the invention.

We claim:

1. A graphical user interface for the documentation of an administered anesthetic drug and the display of an associated pharmacokinetic model and an associated pharmacodynamic model, the graphical user interface comprising:
a first window that upon entry of drug administration data by a clinician, displays the drug administration data, the drug administration data comprising at least a drug name, a drug concentration, an administration type, and an administration time;
a second window displaying a pharmacokinetic model and a pharmacodynamic model, the second window being separate and distinct from the first window;
wherein the pharmacokinetic model and the pharmacodynamic models are overlaid and displayed simultaneously and the pharmacokinetic model and the pharmacodynamic model are based on the drug administration data displayed in the first window.

2. The graphical user interface of claim 1 further comprising a document pre-med reminder, the document pre-med reminder being displayed in the second window when no drug administration data is displayed in the first window.

3. The graphical user interface of claim 2 wherein the document pre-med reminder is displayed in the second window when no pharmacokinetic models and pharmacodynamic models are displayed in the second window.

4. The graphical user interface of claim 1 wherein the second window further comprises a single normalized scale for both the pharmacokinetic model and the pharmacodynamic model.

5. The graphical user interface of claim 4 further comprising a cursor wherein if the cursor is placed at any point on the pharmacokinetic model, a pop-up box is displayed, the pop-up box comprising drug effect site concentration data for the point on which the cursor is placed.

6. The graphical user interface of claim 4 further comprising a touch-sensitive display for displaying the first window and the second window, wherein when a portion of the touch-sensitive display displaying the pharmacokinetic model is touched, a pop-up box is displayed, the pop-up box comprising drug effect site concentration data for the portion of the pharmacokinetic model that is touched.

7. The graphical user interface of claim 4 further comprising a input means for the clinician to select elements within the graphical user interface, wherein upon selection of a position of the pharmacokinetic model, a pop-up box is displayed, the pop-up box comprising drug effect site concentration data for the position of the pharmacokinetic model that is selected.

8. The graphical user interface of claim 1 wherein a clinician may edit any of the drug administration data after it has been displayed in the first window.

9. The graphical user interface of claim 8 wherein the administration type is selected from a list comprising: a bolus and an infusion.

10. The graphical user interface of claim 9 wherein the drug name is the generic drug name.

11. A graphical user interface for the documentation and display of the administration and effect of anesthetic drugs to a clinician, the graphical user interface comprising:
a drug administration window that displays drug administration data representing the administration of a drug by a clinician; and
at least one pharmacokinetic and pharmacodynamic graph display window that displays a pharmacokinetic graph and a pharmacodynamic graph based on the drug administration data displayed in the drug administration window;
wherein a separate pharmacokinetic graph represents each of the drugs administered by the clinician and the pharmacodynamic graph represents the aggregate pharmacodynamic effect of all of the drugs administered by the clinician.

12. The user interface of claim 11, wherein the pharmacokinetic model and the pharmacodynamic models are overlaid and displayed simultaneously with each other and the drug administration data.

13. The user interface of claim 11, wherein the drug administration data comprises a drug name, a drug concentration, an administration type, and an administration time.

14. The user interface of claim 13, wherein the drug name comprises both the common drug name and the generic drug name.

15. The user interface of claim 11, further comprising a document pre-med reminder, the document pre-med reminder being displayed in the graph display window when no drug administration data is displayed in the first window.

16. The user interface of claim 11 wherein the graph display window further comprises a single normalized scale for both the pharmacokinetic model and the pharmacodynamic model.

17. A graphical user interface for the documentation and display of the administration and effect of anesthetic drugs to a clinician, the graphical user interface comprising:
a drug administration window disposed to display drug administration data representing the administration of a drug by a clinician, the drug administration data comprising the time of the administration; and
at least one graph display window that displays at least one pharmacokinetic graph and a pharmacodynamic graph based on the drug administration data displayed in the drug administration window;
wherein the clinician can enter the drug administration data into the user interface retroactively by entering the time of the administration.

18. The graphical user interface of claim 17, wherein the at least one pharmacokinetic graph and the pharmacodynamic graph are overlaid and displayed simultaneously.

19. The graphical user interface of claim 18, wherein the at least one graph display window further comprises a single normalized scale for both the pharmacokinetic graph and the pharmacodynamic graph.

20. The graphical user interface of claim 17, further comprising a cursor wherein if the cursor is placed at any point on the pharmacokinetic graph, a pop-up box is displayed, the pop-up box comprising drug effect site concentration data for the point on which the cursor is placed.
